(19) United States
(12) Patent Application Publication
(10) Pub. No.: US 2006/0264816 A1
(43) Pub. Date: Nov. 23, 2006

(54) Breast pump assembly

(76) Inventor: Brian H. Silver, Cary, IL (US)

Correspondence Address: Michael H. Baniak;BANIAK PINE & GANNON, Suite 1200, 150 N. Wacker Drive, Chicago, IL, 60201, US

(21) Appl. No.: 11/481,180

(22) Filed: Jul. 5, 2006

Related U.S. Application Data
(63) Continuation of application No. 10/285,873, filed on Nov. 1, 2002, now abandoned.

(51) Int. Cl. A61M 1/06

(52) U.S. Cl. 604/74

(57) ABSTRACT

A breast milk pump assembly for use in connection with electric or manual vacuum sources including an improved breast shield having a funnel portion including a heating element for warming the breast shield, enhancing the comfort of the user, and aiding in the expression of the milk from the mammary glands.

### FIELD OF THE INVENTION

The present invention relates generally to a breast milk pump, and more particularly relates to an improved breast pump assembly having a funnel portion including a heating element for warming the breast shield, enhancing the comfort of the user, and aiding in the expression of the milk from the mammary glands.

### BACKGROUND OF THE INVENTION

Generally, breast pumps are well known and usually include a hood or shield that receives the human female breast, a vacuum source connected to the shield for generating and intermittent vacuum (or negative pressure) within the shield, and a receptacle container for the expressed milk. The intermittent suction action of the vacuum source serves to pull on the breast and nipple and thereby extract milk. The extracted milk typically flows from the shield into a collection container for storage and later use.

There is a need in the art for a breast pump assembly including various aids for comforting the user and promoting the expression of milk.

### SUMMARY OF THE INVENTION

The present invention is directed to a breast pump assembly for expressing milk from the mammary glands of the human female. The breast pump assembly includes a breast shield, a container and a vacuum source. The breast shield includes a funnel portion, having a flared element and a tubular element.

The funnel portion includes a heating element for warming the breast shield assembly prior to receiving the breast. A vacuum chamber is in communication with the funnel portion and an outlet to define a passageway. The heating element enhances the comfort of the user and aids in the expression of the milk from the mammary glands.

### BRIEF DESCRIPTION OF THE DRAWINGS

In the course of the detail description, the reference will frequently be made to the attached drawings in which:

FIG. 1 is a perspective illustration of one embodiment of the new improved breast pump assembly constructed in accordance with the principles of the present invention;

FIG. 2 is a perspective view of the foregoing embodiment of the present invention as shown in FIG. 1 attached to a pumping mechanism;

### DETAILED DESCRIPTION OF AN EMBODIMENT OF THE INVENTION

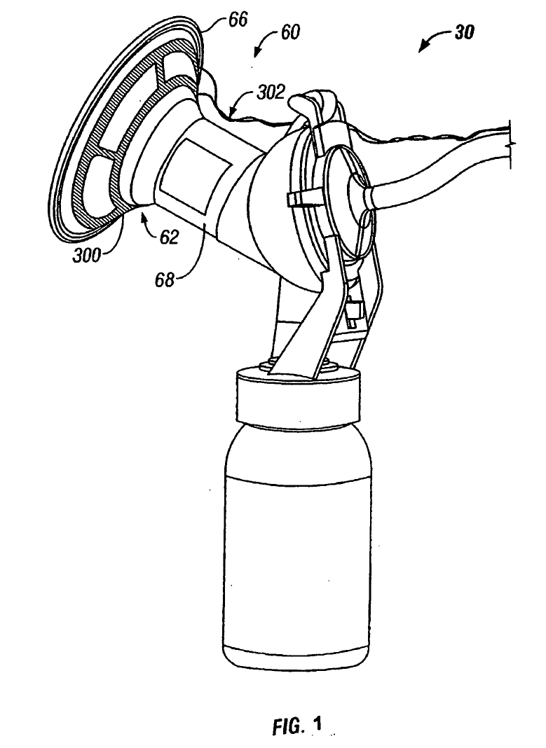

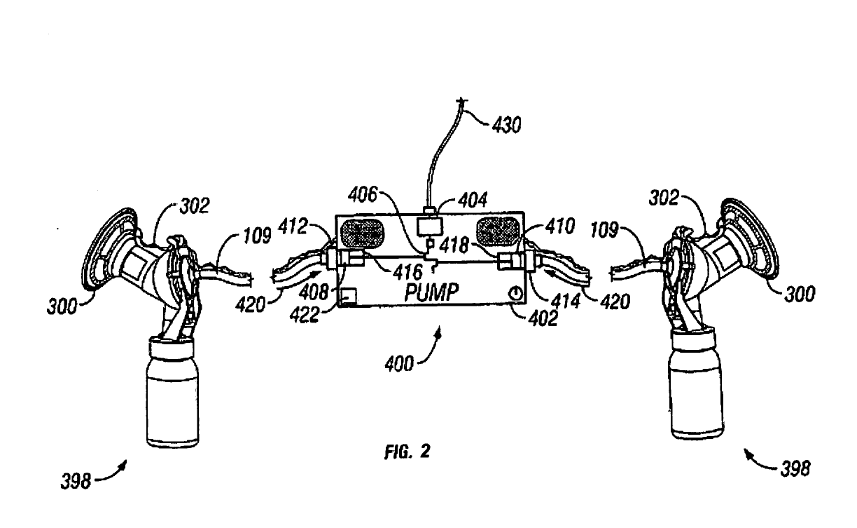

FIG. 1 is a perspective view of an embodiment of the breast pump assembly constructed in accordance with the principles of the present invention. In this embodiment, a heating element 300 is provided on the flared element 66 of the funnel portion 62 of the breast shield assembly 60. Control leads 302 are connected to the heating element 300 and extends to a control device operatively associated with the electric vacuum pump as discussed in FIG. 2. The heating element 300 is preferably an electrically resistive element, or any other suitable device useful for warming the material of the breast shield 60. It will be recognized by those of skill in the art that the breast shield 60 may be formed from any suitable material of construction approved for use in contact with human skin. Preferably the breast shield 60 is formed from a plastic material and may be silicone-based.

It will be further recognized by those of skill in the art that the heating element 300 may be disposed on the flared element 66, tubular element 68 or a combination of the two. Furthermore, the heating element may be disposed on the funnel portion on the exterior surface thereof, embedded within the material or on the interior surface thereof. Preferably, for ease of construction, the heating element 300 is formed on the exterior surface of the funnel portion 62.

The heating element 300 is particularly useful for warming the breast shield prior to receiving the breast which enhances the comfort of the user. Furthermore, the warmed breast shield aids in the expression of milk from the mammary glands and provides therapeutic value. The increased temperature opens the milk ducts and enhances release of the milk in the engorged breast. It is within the teaching of this invention that the heating element 300 may be a device which increases the temperature of breast shield or any other suitable device which achieves the functionality discussed above.

In this embodiment, the breast shield may further include an insert complimentary configured to the funnel portion 62 and received therein for adapting the breast shield 60 for use with differently configured breasts. It will be recognized by those of skill in the art that the heating element 300 will also warm the insert to achieve the above described advantages.

FIG. 2 is a schematic representation of a pair of breast pumps assemblies 30, as described above with respect to FIG. 1 connected to a vacuum source configured as an electric pump 400. The pump 400 has a housing 402 for enclosing a motor 404 which is connected to a crank shaft 406 for reciprocating a pair of pistons 408, 410. Each piston is independently in communication with a port 412, 414 which is each independently in communication with one of the breast shield assemblies 398.

A vacuum is generated when the respective piston 408, 410 is withdrawn in the cylinder 416, 418 and accordingly pulls the air in the supply tube 109 in the direction of arrow 420.

Control leads 302 extend from the heating element 300 on each breast shield assembly 398 to the pump housing 402, wherein the control leads 302 are connected to a control device 422 which allows the user to adjust the intensity of the heating element 300. It will be recognized by those with skill in the art that the structure and function of the control device 422 will vary in accordance with the type of heating element 300 used.

The embodiment described above is illustrative and not restrictive. A scope of the invention is indicated by the claims rather than the foregoing description. The invention has been described in all foreseeable embodiments. Accordingly, all changes which come within the scope of the claims are intended to be embraced therein.

What is claimed is:

1. A breast shield assembly for use with a vacuum source, comprising:
a funnel portion in which a breast is adapted to be received including a heating element formed on the funnel portion for warming the breast shield assembly prior to receiving the breast such that after receiving the breast the milk ducts will open more easily and release of milk from the engorged breast is enhanced;
a vacuum chamber in communication with the funnel portion and an outlet to define a passageway; and
a container for collection of expressed milk.

2. The breast shield assembly as recited in claim 1, wherein the heating element includes a resistive element responsive to a remotely disposed control device.
